(19) United States
(12) Patent Application Publication
(10) Pub. No.: US 2019/0330248 A1
(43) Pub. Date: Oct. 31, 2019

(54) ORGANIC OR INORGANIC METAL HALIDE PEROVSKITES VIA CATION EXCHANGE

(71) Applicant: Iowa State University Research Foundation, Inc., Ames, IA (US)

(72) Inventors: Matthew G. Panthani, Ames, IA (US); Matthew Isaias Milot, Ames, IA (US); Madeleine Kennedy Wilsey, St. Johns, MI (US); Alex James Verburg, Mantorville, MN (US); Atefe Hadi, Ames, IA (US); Umar Hamdeh, Wichita, KS (US); Rainie Nelson, Ames, IA (US)

(21) Appl. No.: 16/282,879

(22) Filed: Feb. 22, 2019

Related U.S. Application Data
(60) Provisional application No. 62/662,974, filed on Apr. 26, 2018.

(51) Int. Cl. C07F 7/24; H01L 51/00

(52) U.S. Cl. CPC C07F 7/24; H01L 51/0032

(57) ABSTRACT

Various embodiments disclosed related to organic or inorganic metal halide perovskites formed via cation exchange and photovoltaic applications thereof. The present invention provides a method of forming an organic or inorganic metal halide perovskite including cation exchanging a hydrocarbylammonium metal halide with a salt comprising an organic or inorganic cation that exchanges with the hydrocarbylammonium cation of the hydrocarbylammonium metal halide, to form the organic or inorganic metal halide perovskite.

### CROSS-REFERENCE TO RELATED APPLICATION

This application claims the benefit of priority to U.S. Provisional Patent Application Ser. No. 62/662,974 filed Apr. 26, 2018, the disclosure of which is incorporated herein in its entirety by reference.

### STATEMENT OF GOVERNMENT SUPPORT

This invention was made with Government support under Contract No. DE-AC02-07CH11358 awarded by Department of Energy, and under Grant No. HRD1619654 awarded by the National Science Foundation. The U.S. Government has certain rights in this invention.

### BACKGROUND

Perovskite refers to minerals having the same crystal structure as calcium titanium oxide (CaTiO₃), known as the perovskite structure, or ^{XII}A^{2+VI}B⁴⁺X²⁻₃ with the oxygen in the face centers. Perovskite solar cells have been an area of interest in emerging solar technologies since 2009, with perovskites such as methylammonium lead halides and methylammonium tin halides. With certified power conversion efficiency (PCE) increasing from about 1% to 22% in 7 years, perovskite cells have become competitive with the PCE of current silicon based solar cells. However, whereas silicon based solar cells have all but reached their theoretical PCE limit, the tunable nature of perovskite solar cells allows for a theoretical limit of around 34%. Additionally, perovskite cells offer the advantage of being partially transparent flexible thin films, with less material being used, potentially saving costs. The transparent nature allows for tandem cells, potentially further boosting PCE. Current tandem cells are so prohibitively expensive that their use has been limited to niche applications such as the aerospace industry, where the main cost driver is weight and cost of fuel. The expense of perovskites offers the possibility of tandem cells that are actually competitive on a cost/watt basis with single crystal silicon.

Typical processing techniques of organolead mixed halide perovskites require a high temperature process including dissolution of the individual single halide species before deposition of thin films (most commonly by spin coating or film casting). The precipitated species have questionable homogeneity and require solvents that include regulated VOCs. Evaporation of these “air toxic” solvents has been a limiting factor for scale-up of solution-based methods.

### SUMMARY OF THE INVENTION

Various embodiments of the present invention provide a method of forming an organic or inorganic metal halide perovskite. The method includes cation exchanging a hydrocarbylammonium metal halide with a salt including an organic or inorganic cation that exchanges with the hydrocarbylammonium cation of the hydrocarbylammonium metal halide, to form the organic or inorganic metal halide perovskite, wherein the hydrocarbyl group is substituted or unsubstituted.

Various embodiments of the present invention provide a method of forming a methylammonium lead iodide perovskite film. The method includes melt-processing phenylethylammonium lead iodide at a temperature of no greater than 295° C., to form a melt-processed phenylethylammonium lead iodide film. The method also includes cation exchanging the melt-processed phenylethylammonium lead iodide film with a solution of methylammonium iodide that exchanges with the phenylethylammonium cation of the phenylethylammonium lead iodide film, to form the methylammonium lead iodide perovskite film.

Various embodiments provide an organic or inorganic metal halide perovskite film formed via the method, which can be used to form photovoltaic devices such solar cells. Various embodiments of the present invention provide a method of forming a photovoltaic device including the organic or inorganic metal halide perovskite.

In various embodiments, the method of the present invention provides certain advantages over other methods of forming organic or inorganic metal halides. For example, in some embodiments, the method of the present invention of forming the organic or inorganic metal halide perovskite can be performed near the melting temperature and below the decomposition temperature of the materials used. In various embodiments, the method can form higher quality organic or inorganic metal halide films, such as having shorter electron and hole diffusion lengths, allowing production of photovoltaic devices having higher conversion efficiencies. In various aspects, the organic or inorganic metal halide films are more stable than organic or inorganic metal halide films produced via other methods, such as due to residues of hydrocarbylammonium metal halide remaining after the cation exchange.

In various embodiments, the method of the present invention includes cation exchanging an exchangeable melt-processed hydrocarbylammonium metal halide film such as phenylethylammonium metal halide with organic cation exchange to produce films of organic or inorganic metal halide, a material which cannot be effectively formed directly via melt-processing due to the high temperatures required. By first producing another compound that can be easily melt-processed, and transforming that compound into the desired organic or inorganic metal halide, the organic or inorganic metal halide can enjoy the advantages of melt-processing such as freedom from significant organic solvents and evaporation thereof, easy scale-up, avoidance of high temperatures, and potentially higher quality perovskite films such as having enhanced photovoltaic utility.

### BRIEF DESCRIPTION OF THE FIGURES

The drawings illustrate generally, by way of example, but not by way of limitation, various embodiments of the present invention.

FIG. 1A illustrates a photograph of PEA₂PbI₄ crystals, in accordance with various embodiments.

FIG. 1B illustrates a photograph of (PEA)₂SnI₄ crystals, in accordance with various embodiments.

FIG. 2 illustrates a procedure for melt processing to form phenylethylammonium lead iodide films, in accordance with various embodiments.

FIG. 3A illustrates a photograph of melt-processed phenylethylammonium lead iodide film, in accordance with various embodiments.

FIG. 3B illustrates a photograph of melt-processed phenylethylammonium tin iodide film, in accordance with various embodiments.

FIG. 4 illustrates a procedure for dip processing melt-processed phenylethylammonium lead iodide films to form methylammonium lead iodide films, in accordance with various embodiments.

FIG. 5 illustrates a methylammonium lead iodide film formed via dip-processing, in accordance with various embodiments.

FIG. 6 illustrates a powder X-ray diffraction spectrum for the phenylethylammonium lead iodide crystals, in accordance with various embodiments.

FIG. 7 illustrates an X-ray diffraction spectrum of the dip-processed methylammonium lead iodide film of Example 2, in accordance with various embodiments.

FIG. 8 illustrates a UV-vis spectrum shown absorbance versus wavelength for melt-processed phenylethylammonium lead iodide and dip-processed methylammonium lead iodide, in accordance with various embodiments.

FIG. 9 illustrates an FTIR spectrum showing intensity versus wavenumber for phenylethylammonium lead iodide crystals, in accordance with various embodiments.

FIG. 10 illustrates an FTIR spectrum showing absorbance versus wavelength for (β-Me-PEA)₂PbI₄ and cation-exchanged (MA)Pb₃, in accordance with various embodiments.

FIGS. 11A-F illustrate scanning electron microscopy images of (β-Me-PEA)₂PbI₄ melted onto a 1-inch substrate, in accordance with various embodiments.

FIGS. 12A-B illustrate scanning electron microscopy images of (β-Me-PEA)₂PbI₄ melted onto a ¼-inch substrate, in accordance with various embodiments.

FIG. 13 illustrates an X-ray diffraction spectrum of (β-Me-PEA)₂PbI₄ melted onto a ¼-inch substrate and onto a 1-inch substrate, in accordance with various embodiments.

FIG. 14 illustrates PEA₂PbI₄ crystals, PEA₂SnI₄ crystals, and PEA₂(Sn₁₋ₓPbₓ)I₄ crystals having varying amounts of Sn and Pb, in accordance with various embodiments.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y. or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ—C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

As used herein, the term “polymer” refers to a molecule having at least one repeating unit and can include copolymers.

In various embodiments, salts having a negatively charged counterion can include any suitable negatively charged counterion. For example, the counterion can be a halide, such as fluoride, chloride, iodide, or bromide. In other examples, the counterion can be nitrate, hydrogen sulfate, dihydrogen phosphate, bicarbonate, nitrite, perchlorate, iodate, chlorate, bromate, chlorite, hypochlorite, hypobromite, cyanide, amide, cyanate, hydroxide, permanganate. The counterion can be a conjugate base of any carboxylic acid, such as acetate or formate. In some embodiments, a counterion can have a negative charge greater than −1, which can in some embodiments complex to multiple ionized groups, such as oxide, sulfide, nitride, arsenate, phosphate, arsenite, hydrogen phosphate, sulfate, thiosulfate, sulfite, carbonate, chromate, dichromate, peroxide, or oxalate.

### Method of Forming an Organic or Inorganic Metal Halide Perovskite.

Various embodiments of the present invention provide a method of forming an organic or inorganic metal halide perovskite. The method includes cation exchanging a hydrocarbylammonium metal halide with a salt, such as using a solution of the salt that is exposed to the hydrocarbylammonium metal halide. The salt includes an organic or inorganic cation with a suitable anionic counterion. The cation exchange includes exchanging the organic or inorganic cation with the hydrocarbylammonium cation of the hydrocarbylammonium metal halide, to form the organic or inorganic metal halide perovskite. The hydrocarbyl group can be substituted or unsubstituted.

The organic or inorganic metal halide perovskite can be in any suitable physical form. For example, the organic or inorganic metal halide can be in the form of particles (e.g., a dispersion of particles, or a coating or layer of particles), or in the form of a monolithic shape such as a film. The method can be a method of forming an organic or inorganic metal halide perovskite film, wherein the hydrocarbylammonium metal halide that is cation exchanged is a hydrocarbylammonium metal halide film.

The film can be a thin film, having an average thickness of about 1 nm to about 10 mm, about 350 nm to about 950 nm, about 500 nm to about 800 nm, or about 1 nm or less, or less than, equal to, or greater than 2 nm, 3, 4, 5, 6, 8, 10, 15, 20, 25, 50, 100, 150, 200, 250, 300, 350, 400, 450, 500, 550, 600, 650, 700, 750, 800, 850, 900, 950 nm, 1 micron, 2, 3, 4, 5, 10, 15, 20, 25, 50, 100, 150, 200, 250, 500, 750 microns, 1 mm, 2, 3, 4, 5, 6, 7, 8, 9, or about 10 mm or more. The method can include forming the organic or inorganic metal halide perovskite in any suitable environment. In an embodiment, the method includes performing the cation exchange of the hydrocarbylammonium metal halide while the hydrocarbylammonium metal halide is on a substrate, such that the produced organic or inorganic metal halide is also on the substrate.

The hydrocarbylammonium metal halide can be any suitable hydrocarbylammonium metal halide, such as having the chemical formula (R—NH₃)₂MX₄ or [R—NH₃]⁺₂[MX₄]⁻, wherein [R—NH₃]⁺ is the hydrocarbylammonium cation and [MX₄]⁻ is a metal atom bonded to four halide atoms that are the same or different and having a negative charge. The metal can be any suitable metal that forms the organic or inorganic metal halide as described herein, such as lead or tin, or a combination thereof (e.g., M=Sn₁₋ₓPbₓ, wherein x is greater than 0 and less than 1).

In some embodiments, the organic or inorganic metal halide can have the structure ZMX₃ or [Z]⁺[MX₃]⁻. The variable Z is the organic or inorganic cation. The organic or inorganic metal halide can be an organic or inorganic lead halide, an organic or inorganic tin halide, or a combination thereof. The organic or inorganic metal halide can be an organic or inorganic metal iodide, an organic or inorganic metal bromide, an organic or inorganic metal chloride, an organic or inorganic metal halide comprising a mixture of two or more halides, or a combination thereof. The organic or inorganic metal halide can be an organic or inorganic lead iodide, an organic or inorganic lead bromide, an organic or inorganic lead chloride, an organic or inorganic lead halide comprising a combination of two or more halides, an organic or inorganic tin iodide, an organic or inorganic tin bromide, an organic or inorganic tin chloride, an organic or inorganic tin halide comprising a combination of two or more halides, or a combination thereof.

In embodiments wherein the salt is a methylammonium salt, the methylammonium metal halide can be any suitable methylammonium metal halide, such as having the chemical formula (CH₃—NH₃)MX₃ or [CH₃—NH₃]⁺[MX₃]⁻, wherein [CH₃—NH₃]⁺ is the methylammonium cation and [MX₃]⁻ is a metal atom bonded to three halide atoms that are the same or different and having a negative charge. The metal can be any suitable metal that provides a perovskite structure, such as lead or tin or a combination thereof (e.g., Sn₁₋ₓPbₓ); the methylammonium metal halide can be a methylammonium lead halide, a methylammonium tin halide, or a combination thereof. The methylammonium metal halide can be a methylammonium metal iodide, methylammonium metal bromide, a methylammonium metal chloride, a methylammonium metal halide comprising a mixture of two or more halides, or a combination thereof. The methylammonium metal halide can be a methylammonium lead iodide, methylammonium lead bromide, a methylammonium lead chloride, a methylammonium lead halide comprising a combination of two or more halides, a methylammonium tin iodide, methylammonium tin bromide, a methylammonium tin chloride, a methylammonium tin halide comprising a combination of two or more halides, or a combination thereof. The methylammonium metal halide can be a methylammonium lead iodide (CH₃—NH₃)PbI₃, a methylammonium tin iodide (CH₃—NH₃)SnI₃, or a methylammonium tin-lead iodide (CH₃—NH₃)(Sn₁₋ₓPbₓ)I₃.

### Cation Exchanging.

The method includes cation exchanging a hydrocarbylammonium metal halide with a salt including an organic or inorganic cation. The cation exchange includes exchanging the organic or inorganic cation with the hydrocarbylammonium cation of the hydrocarbylammonium metal halide, to form the organic or inorganic metal halide perovskite.

The organic or inorganic cation in the salt can be any suitable cation that exchanges with the hydrocarbylammonium cation as described herein. For example, the cation can be cesium, rubidium, formamidinium, potassium, methylammonium, or a combination thereof. The method can be a method of forming a methylammonium metal halide perovskite, wherein the cation is methylammonium. The salt can be any suitable salt, such as methylammonium iodide, methylammonium chloride, methylammonium bromide, or a combination thereof.

The cation exchange can be performed in any suitable way, such that the organic or inorganic cation exchanges with the hydrocarbylammonium cation to form the organic or inorganic metal halide perovskite. The cation exchange can be performed in solution, using a suitable solvent that adequately dissolves both the organic or inorganic salt and allows the cation exchange to occur. The solvent can be an organic solvent, such as a polar organic solvent, which can optionally be degassed. the solvent can be an organic alcohol, such as a (C₁-C₅)alcohol, such as isopropanol.

The cation exchange can be performed under any suitable conditions. For example, the cation exchange can include placing the hydrocarbylammonium metal halide in a solution including the salt, with optional agitation, at sufficient temperature and for sufficient time such that a predetermined proportion of the hydrocarbylammonium metal halide is cation exchanged to form the organic or inorganic metal halide. The temperature used can be 0° C. to 100° C., 10° C. to 50° C., or less than, equal to, or greater than 0° C., 10, 20, room temperature, 30, 40, 50, 60, 70, 80, 90, or about 100° C. or more. The temperature used can be less than the decomposition temperature of the materials used; for example, methylammonium lead iodide starts to decompose at about 60° C., and the temperatures used during the cation exchanging can be less than 60° C. The time can be about 1 second to about 1 week, 30 seconds to 10 minutes, or about 1 second or less, or less than, equal to, or greater than 30 seconds, 1 minute, 2, 3, 4, 5, 10, 15, 20, 30, 40, 50 minutes, 1 hour, 2, 3, 4, 5, 10, 15, 20 hours, 1 day, 2, 3, 4, 5, 6 days, or about 1 week or more. The proportion of the hydrocarbylammonium metal halide that is converted to the organic or inorganic metal halide can be any suitable proportion, such as about 0.001 mol % to about 100 mol %, about 10 mol % to about 90 mol %, or about 0.001 mol % or less, or less than, equal to, or greater than about 0.01 mol %, 0.1, 1, 2, 3, 4, 5, 6, 8, 10, 12, 14, 16, 18, 20, 25, 30, 35, 40, 45, 50, 55, 60, 65, 70, 75, 80, 85, 90, 91, 92, 93, 94, 95, 96, 97, 98, 99, 99.9, 99.99, or about 99.999 mol % or more. In various aspects, thinner films provide more complete conversion to the organic or inorganic metal halide. In various aspects, conversion can occur beyond the surface of the film and can include interior regions of the film that are accessible via the cation exchange process.

The cation exchanging can optionally include annealing or drying the formed organic or inorganic metal halide at a suitable temperature and time, such as at about 50-100° C., for about 10 seconds to about 5 minutes, such that the organic or inorganic metal halide is sufficiently formed and dried

### Melt-Processing.

The hydrocarbylammonium metal halide that is cation exchanged can be any suitable hydrocarbylammonium metal halide that can be effectively cation exchanged. The hydrocarbylammonium metal halide can be a film, which is cation exchanged to form an organic or inorganic metal halide film. The hydrocarbylammonium metal halide can be a melt-processed hydrocarbylammonium metal halide film, wherein melt-processing including heating hydrocarbylammonium metal halide crystals suitably to form the hydrocarbylammonium metal halide film, such as including heating suitably to melt the hydrocarbylammonium metal halide crystals.

Melt-processing to form a melt-processed hydrocarbylammonium metal halide film can include heating hydrocarbylammonium metal halide to a temperature of no greater than about the melting point of the hydrocarbylammonium metal halide, to form the melt-processed hydrocarbylammonium metal halide film. For example, phenylethylammonium lead iodide melts at about 252.9° C. The melt-processing can include heating the hydrocarbylammonium metal halide to a temperature of no greater than about 300° C., no greater than about 295° C., no greater than about 291° C., or about 250° C. to about 300° C., or about 270° C. to about 295° C., or about 250° C. or less, or less than, equal to, or greater than 252° C., 254, 256, 258, 260, 262, 264, 266, 268, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299° C., or about 300° C. or more. The melt-processing can include applying heat on one face of the film, and simultaneously applying heat on the other face of the film, such as using hot plates or other suitable sources of heat, wherein each heat can be the same or different and is independently selected from the temperatures in the preceding sentence. The heat can be directly applied by contacting the heating source and the film, or the heat can be applied via indirect contact such as using a substrate (e.g., a glass or Teflon substrate) to separate the film and the heating source. Indirect application of heat can help to avoid sticking of the film to the heating source once the heating source is removed. Application of heat from two or more different sources, such as on the top and bottom of the film, can include using indirect application of heat via a substrate on both faces of the film.

The hydrocarbylammonium group in the hydrocarbylammonium metal halide can be any suitable hydrocarbylammonium group such that the organic or inorganic cation of the salt exchanges with the hydrocarbylammonium cation to form the organic or inorganic metal halide perovskite. Unless otherwise indicated, a hydrocarbyl group can be substituted or unsubstituted. The hydrocarbylammonium group can be a phenyl(C₁-C₅)alkylammonium group, wherein the phenyl is unsubstituted or substituted with one or more halides. The hydrocarbylammonium group can be a phenylethylammonium group, wherein the phenyl is unsubstituted or substituted with one or more halides. The hydrocarbylammonium group can be phenylethylammonium (PEA), 2-fluorophenethylammonium (2-FPEA), 3-fluorophenethylammonium (3-FPEA), β-methylphenethylammonium (β-Me-PEA), or 2,3,4,5,6-pentafluorophenethylammonium (5FPEA). The hydrocarbylammonium metal halide can be phenylethylammonium lead iodide or phenylethylammonium tin iodide.

### Substrate.

The hydrocarbylammonium metal halide and the organic or inorganic metal halide perovskite can be formed on a substrate, such as formed as a film on the substrate. The substrate can be any suitable substrate that is compatible with embodiments of the method of making the organic or inorganic metal halide. The substrate can be a glass substrate or can include one or more glass layers, such as fluorine doped tin oxide (FTO) glass. The substrate can be a TiO₂ substrate or can include one or more TiO₂ layers, such as including one or more TiO₂ layers coated on a glass substrate. The TiO₂ layers can be or can include TiO₂ nanoparticles. The TiO₂ layers can be formed from TiO₂ paste (e.g., to form mesoporous TiO₂ m-(TiO₂)), or from a reagent such as titanium isopropoxide (e.g., to form compact TiO₂ (c-TiO₂)). In some embodiments, the TiO₂ is spin-coated to form the layer, such as spin coated onto a glass layer or onto another layer. In some embodiments, the substrate is a FTO glass layer, one or more c-TiO₂ layers, and one or more m-TiO₂ layers, wherein the hydrocarbylammonium metal halide and the organic or inorganic metal halide perovskite is formed on the m-TiO₂ layer of the substrate. The substrate can enable the organic or inorganic iodide to function as part of a photovoltaic device.

### Organic or Inorganic Ammonium Metal Halide Perovskite.

In various embodiments, the present invention provides an organic or inorganic metal halide perovskite, such as any organic or inorganic metal halide formed by the method of the present invention including cation exchange. The organic or inorganic metal halide perovskite can be a film, such as a film on a substrate. The organic or inorganic metal halide can be part of a photovoltaic device, or can be formed into a photovoltaic device.

### Photovoltaic Device.

In various embodiments, the present invention provides a method of forming a photovoltaic device, the method including forming an organic or inorganic metal halide film using the method of the present invention including cation exchange, and including forming the photovoltaic device including the organic or inorganic metal halide film. In various embodiments, the present invention provides a photovoltaic device formed by an embodiment of the method of forming a photovoltaic device.

The photovoltaic device can be any suitable photovoltaic device, such as a solar cell, such as a dye-sensitized solar cell. For example, the organic or inorganic metal halide film can function as a dye layer that absorbs sunlight to excite electrons, titanium dioxide can function as an anode that accepts electrons from the dye layer, and a suitable cathode (e.g., platinum) and electrolyte can be used that transports electrons back to the dye layer.

### EXAMPLES

Various embodiments of the present invention can be better understood by reference to the following Examples which are offered by way of illustration. The present invention is not limited to the Examples given herein.

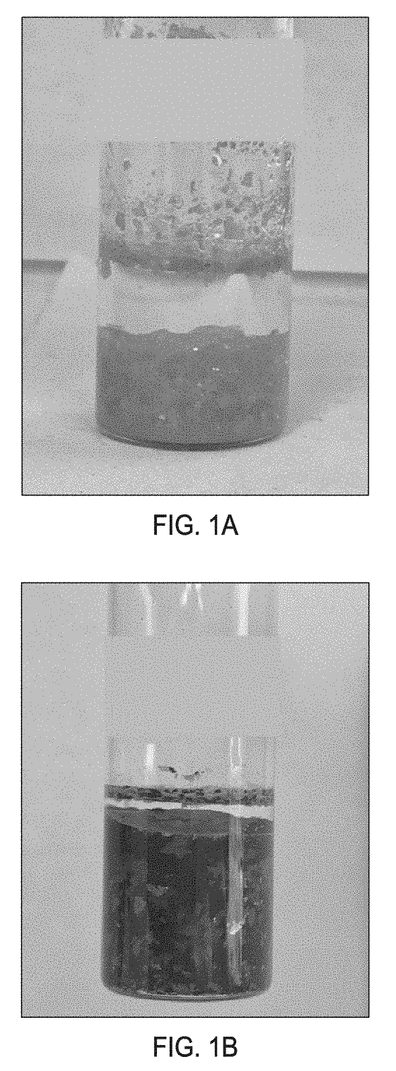

Materials. All chemicals were purchased from Sigma-Aldrich unless stated otherwise. (PEA)₂Pb₄ crystal were synthesized by dissolving stoichiometric amounts of lead oxide (PbO) and phenylethylamine (PEA) in hydroiodic acid solution (HI) (57% w/w). HI solution (12 mL) was added to 1 mmol (0.461 gr) PbO. PEA (0.252 ml, 2 mmol) was added dropwise on top of HI solution. The solution was heated to 130° C. on hotplate while stirring at 750 rpm until all precursors were dissolved and clear yellow solution was obtained. The stir rate was turned off, and the solution was cooled down naturally. Upon cooling, orange plate-like crystals were appeared. Vacuum filtration was used to separate the crystals from HI solution, and the crystals were washed 3 times with copious amounts of diethyl ether to remove any residues of HI. Washed crystals were dried in a vacuum oven overnight and stored in an N₂ filled glovebox. Finally, the PEA₂PbI₄ crystals were ground into a fine powder for melt processing. A photograph of the PEA₂PbI₄ crystals is reproduced in FIG. 1A. A photograph of (PEA)₂SnI₄ crystals made by a similar procedure is reproduced in FIG. 1B; the procedure was the same except tin chloride dehydrate and phenethylammonium iodide were dissolved in hydriodic acid solution and hypophosphorous acid (H₃PO₂) solution (50% w/w). The hypophosphorous acid was added to prevent the oxidation of tin compounds).

### Example 1. Melt Processing to Form Phenylethylammonium Lead Iodide Films

Substrate preparation. There were four layers above the blank glass substrate: 1) fluorine doped tin oxide, 2) compact TiO₂ (0.15 M), 3) compact TiO₂ (0.30 M), and mesoporous TiO₂. Onto fluorine doped tin oxide (FTO) glass was spin-coated at 4500 RPM for 30 seconds 150-200 microliters of a c-TiO₂ (0.15 M) solution (0.8449 mL titanium isopropoxide, 9.155 mL anhydrous ethanol) through a syringe with a 0.45 micron filter. The corners of the substrate were cleaned with ethanol using a cotton swab. Annealing was performed on a hotplate set at 250° C. for 5 minutes. The substrate was allowed to cool. Then, the substrate was placed back on the spin-coater and 150-200 microliters of a second c-TiO₂ (0.30 M) solution (1.687 mL titanium isopropoxide, 8.310 mL anhydrous ethanol) were injected through a syringe with a 0.45 micron filter to spin-coat at 4500 RPM for 30 seconds. The substrate was annealed on a hotplate set at 450° C. for 30 minutes. A m-TiO₂ solution was prepared by dissolving 1 g of TiO₂ paste (Dyesol) with 3.15 g (4.44 mL) of EtOH. The solution was spin-coated onto the c-TiO₂ layer at 4500 RPM for 30 seconds, through a micropipette, and annealed at 450° C. for 30 minutes.

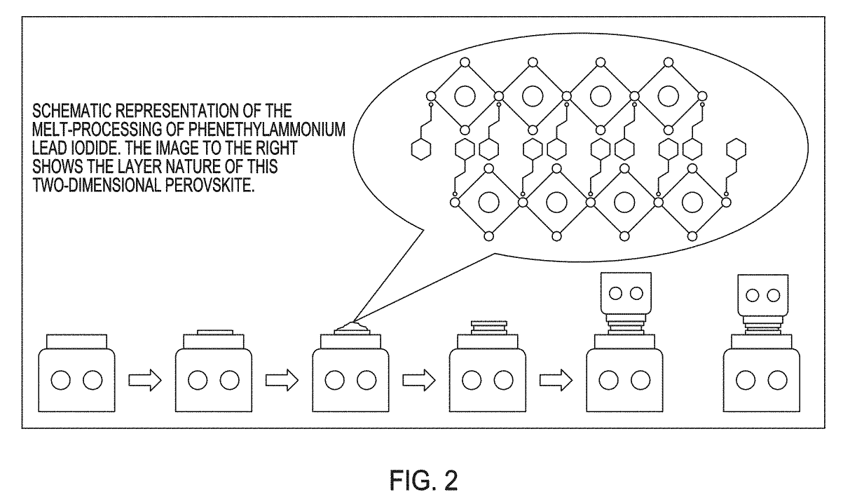

The procedure for melt processing to form phenylethylammonium lead iodide films is shown in FIG. 2. Melt processed films were prepared by evenly spreading 15 mg of finely ground (PEA)₂PbI₄ powder from Example 1 onto the mesoporous TiO₂-coated substrate. The substrate was placed onto a ⅜″ thick aluminum plate atop a hotplate, with the temperature of the aluminum plate set to 275° C. for (PEA)₂PbI₄. A blank glass substrate covered with PTFE tape or film (PTFE does not adhere to the (PEA)₂Pb₄ crystals, and PTFE film avoids melted crystals between the tape and the substrate) was placed on top of the m-TiO₂ substrate with the PTFE tape facing the m-TiO₂ substrate. A separate hot plate set to 291° C. is then placed on top of the blank glass substrate for 20 seconds, melting the crystals on the m-TiO₂ substrate. The top hot plate is removed, the PTFE covered glass substrate is carefully removed from the m-TiO₂ substrate, and the melt processed film is removed from the heat and allowed to cool naturally in ambient conditions.

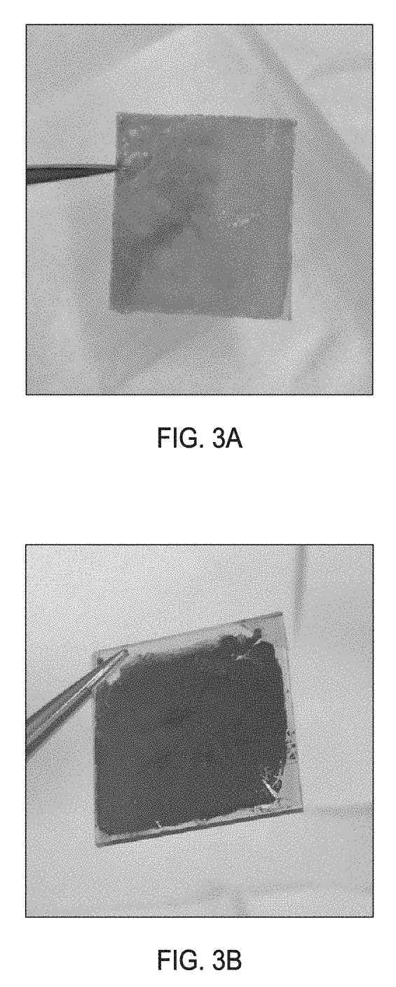

A photograph of the melt-processed phenylethylammonium lead iodide film is shown in FIG. 3A. A photograph of melt-processed phenylethylammonium tin iodide film, made from the (PEA)₂SnI₄ crystals from Example 1 using the same procedure, is reproduced in FIG. 3B.

### Example 2. Methylammonium Lead Iodide Films

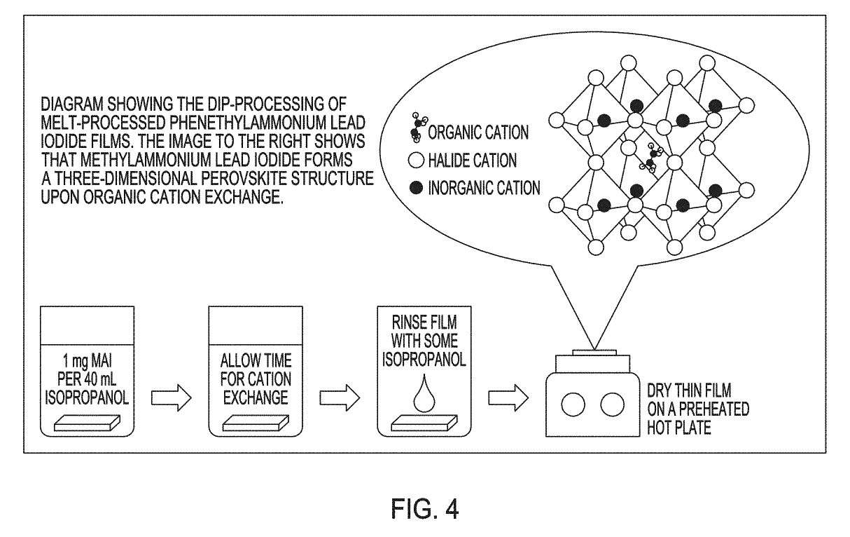

The procedure used for dip processing/cation exchange to form methylammonium lead iodide firms is shown in FIG. 4. MAPbI₃ films derived from melt processed (PEA)₂PbI₄ films were produced via cation exchange. Methylammonium iodide (MAI) powder (400 mg) was dissolved in 40 mL of degassed isopropanol. Melt processed (PEA)₂PbI₄ films were subsequently submerged into the MAI/isopropanol solution for 2 minutes. The film transitioned from an yellow/orange color to a dark brown color indicating the transformation into MAPbI₃. Finally, the films were annealed/dried on a hotplate set to about 80° C. for 1 minute.

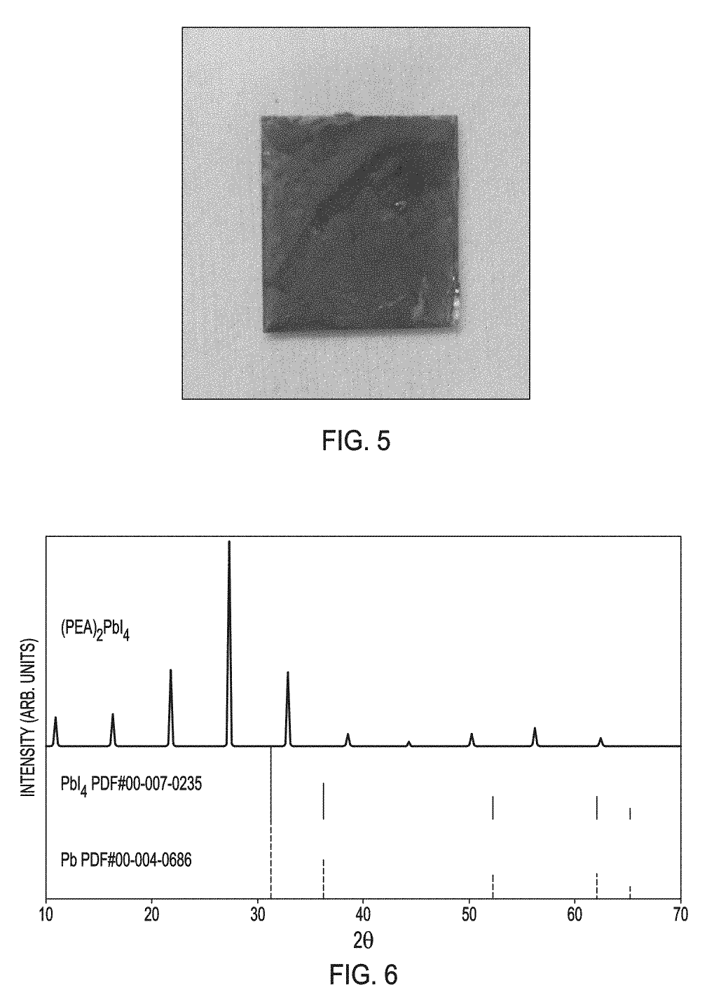

A photograph of the methylammonium lead iodide film is reproduced in FIG. 5.

When submerged in isopropanol solution, phenylethylammonium tin iodide showed a transition from a dark brown color to a gray color which is indicative of light scattering.

### Example 3. Characterization

Characterization Methods. Absorbance, transmittance, and reflectance data were collected with a PerkinElmer Lambda 750 spectrophotometer equipped with a Labsphere 100 mm integrating sphere. XRD of powders and thin films were collected using a Bruker DaVinci D8 Advance diffractometer with a Cu Kα radiation source. A Bruker Tensor 37 with wavenumbers ranging from 6000 cm⁻¹ to 400 cm⁻¹ and with a maximum resolution of 0.5 cm⁻¹ was used to characterize the organic cations of films that underwent cation exchange. An Agilent 6540 QTOF LC-MS was used to characterize the mass of molecules in the film after cation exchange using electrospray ionization (ESI) as the ionization technique.

FIG. 6 illustrates a powder X-ray diffraction spectrum for the phenylethylammonium lead iodide crystals of Example 1, supporting the conclusion that phenylethylammonium lead iodide was synthesized.

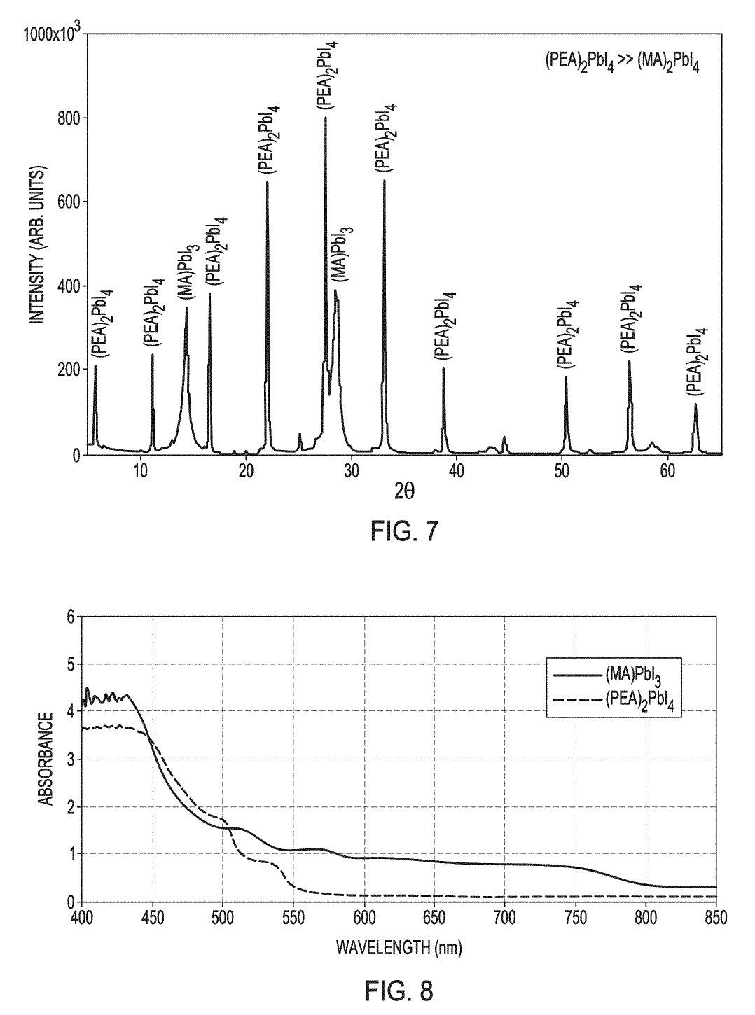

FIG. 7 illustrates an X-ray diffraction spectrum of the dip-processed methylammonium lead iodide film of Example 2, supporting the conclusion that dip-processing is an effective method of synthesizing methylammonium lead iodide.

FIG. 8 illustrates a UV-vis spectrum showing absorbance versus wavelength for melt-processed phenylethylammonium lead iodide and dip-processed methylammonium lead iodide. Methylammonium lead iodide shows characteristic absorptions at around 525 nm and 750 nm.

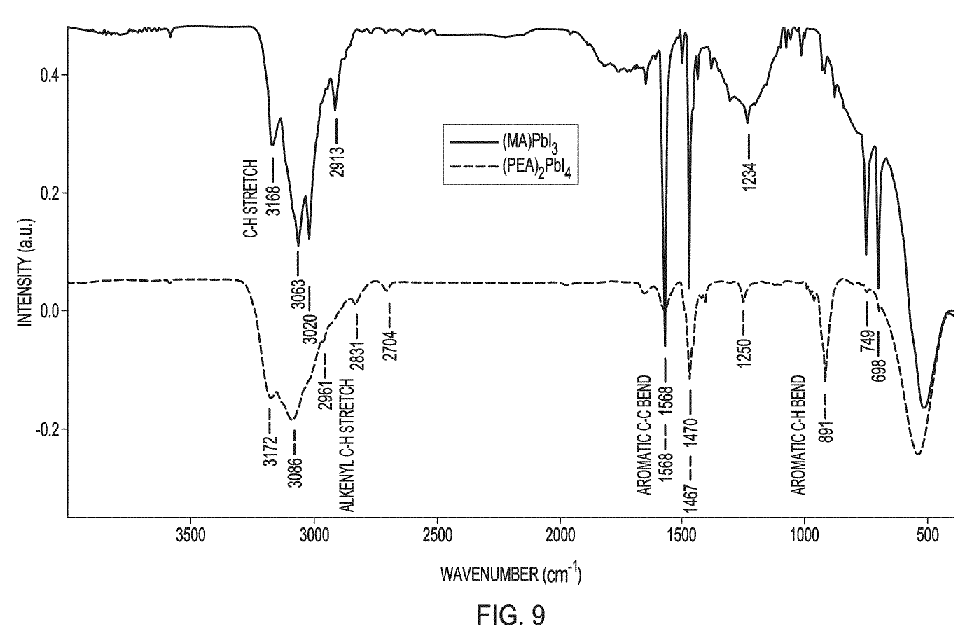

FIG. 9 illustrates an FTIR spectrum showing intensity versus wavenumber for the phenylethylammonium lead iodide crystals of Example 1, and for the dip-processed methylammonium lead iodide film of Example 2. The spectrum supports the conclusion that phenylethylammonium lead iodide was synthesized, and that dip-processing is an effective method of synthesizing methylammonium lead iodide. FIG. 9 illustrates that the peak intensity at 1568 (cm⁻¹) for C═C bond (aromatic ring in PEA) was reduced after MA cation exchange. The changes in the peak shape ˜3010-3100 (cm⁻¹) could imply the difference in the C—H stretch after cation exchange.

### Example 4. Photovoltaic Devices

The PCE of PV devices was determined using current-voltage (J-V) characterization under solar simulation (Newport, M-9119X with an AM1.5G filter). The intensity was adjusted to 100 mW/cm² using an NREL certified Hamamatsu mono-Si photodiode (S 1787-04). All devices were tested inside a N₂-filled glovebox.

### Example 5. Modified Heating/Coating Stage

Instead of using two hotplates, for melt processing to form phenylethylammonium lead iodide films a TBK-318 “3 in 1 glue remove machine” was used. The machine heated the stage and the blade separately. The heating stage was modified to reach the required temperature.

### Example 6. (β-Me-PEA)₂PbI₄ and (MA)PbI₃ Films

The procedures of Example 1 and 2 were following, using the modified heating/coating stage described in Example 5, replacing the PEA group with the less bulky beta-methyl-phenylethylammonium group (Ph-CH(CH₃)—CH₂—NH₃⁺).

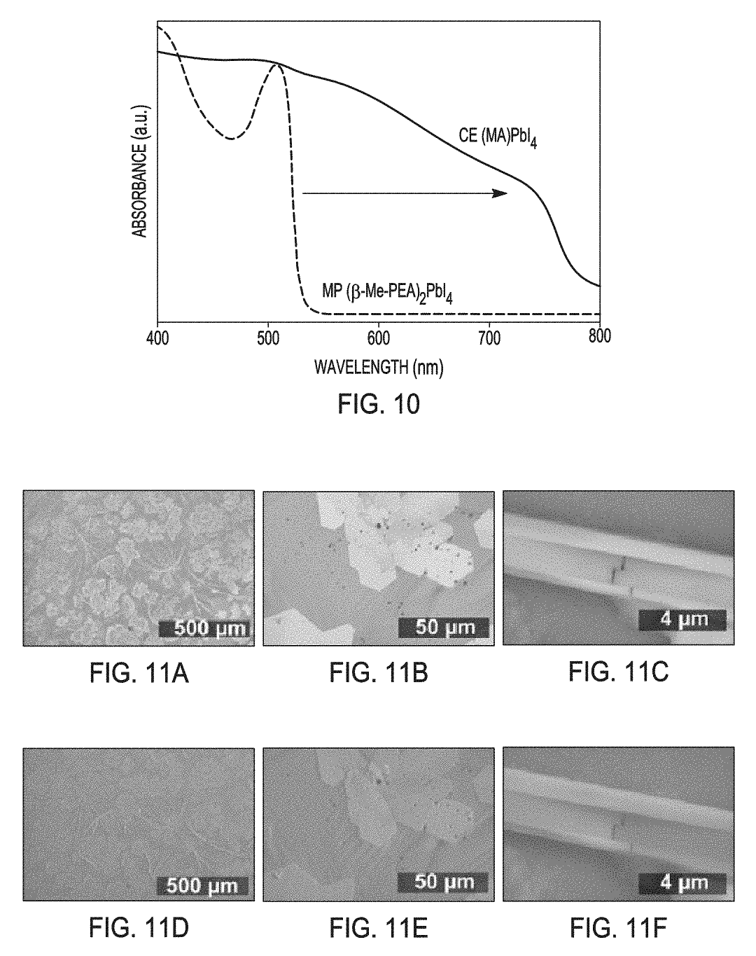

FIG. 10 illustrates an FTIR spectrum showing absorbance versus wavelength for (β-Me-PEA)₂PbI₄ and the cation-exchanged (MA)PbI₃. (MA)PbI₃ demonstrated an absorption peak at 750 nm. (β-Me-PEA)₂PbI₄ demonstrated an absorption peak at 525 nm. The lower band gap inherent in (MA)Pb₃ can be deduced from the spectrum.

### Example 7. Variation of Substrate Size

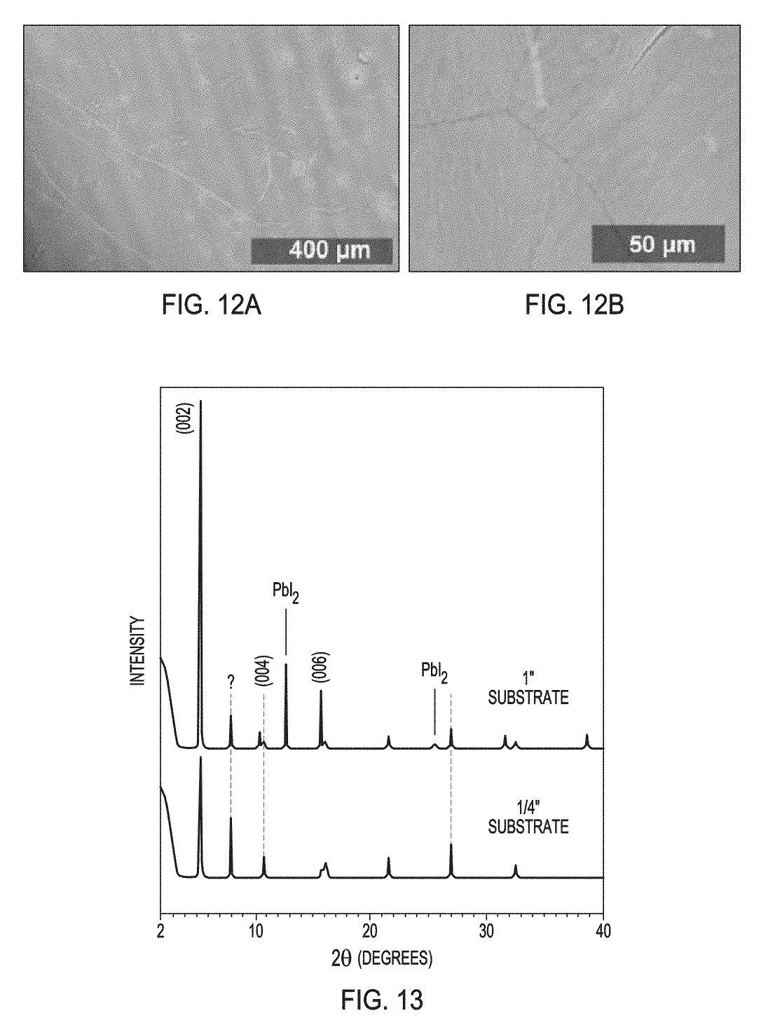

(β-Me-PEA)₂PbI₄ was melted onto two substrate sizes (1″ and ¼″). The substrate size had an impact on the film morphology and degradation of these materials. FIGS. 11A-F illustrate scanning electron microscopy images of (β-Me-PEA)₂PbI₄ melted onto a 1-inch substrate. FIGS. 12A-B illustrate scanning electron microscopy images of (β-Me-PEA)₂PbI₄ melted onto a ¼-inch substrate. The smaller substrate (¼″) had smooth continuous film morphology compared to the larger substrate (1″). FIG. 13 illustrates an X-ray diffraction spectrum of (β-Me-PEA)₂PbI₄ melted onto a ¼-inch substrate and onto a 1-inch substrate, showing that PbI₂ was formed on the larger substrate, with no corresponding peaks for PbI₂ found on the smaller substrate. It is hypothesized that the longer exposure time to elevated temperature (˜207° C.) to coat the larger substrate could evaporate organic cation, which could be corrected by adding an additional amount of organic salt and by reducing the exposure time.

### Example 8. PEA₂(Sn₁₋ₓPbₓ)I₄ Crystals

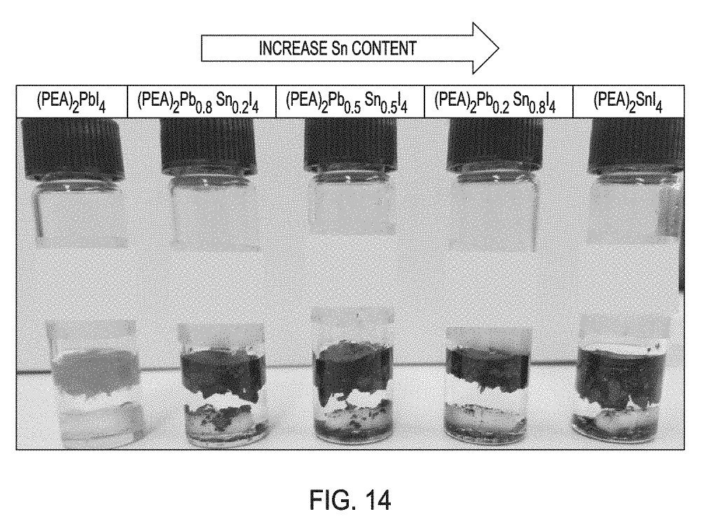

PEA₂(Sn₁₋ₓPbₓ)I₄ crystals were formed. Replacing the B-site cation (Pb with Sn) not only reduces the risk of health issues, but also could reduce the melting temperature. FIG. 14 illustrates PEA₂PbI₄ crystals, PEA₂SnI₄ crystals, and PEA₂(Sn₁₋ₓPbₓ)I₄ crystals having varying amounts of Sn and Pb.

The PEA₂(Sn₁₋ₓPbₓ)I₄ are melt processed to form films using a procedure similar to that of Example 1, and the films are treated using a procedure similar to that of Example 2, to form methylammonium lead iodide films.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Embodiments

The following exemplary embodiments are provided, the numbering of which is not to be construed as designating levels of importance:

Embodiment 1 provides a method of forming an organic or inorganic metal halide perovskite, the method comprising:

cation exchanging a hydrocarbylammonium metal halide with a salt comprising an organic or inorganic cation that exchanges with the hydrocarbylammonium cation of the hydrocarbylammonium metal halide, to form the organic or inorganic metal halide perovskite, wherein the hydrocarbyl group is substituted or unsubstituted.

Embodiment 2 provides the method of Embodiment 1, wherein the cation is cesium, rubidium, formamidinium, potassium, methylammonium, or a combination thereof.

Embodiment 3 provides the method of any one of Embodiments 1-2, wherein the method is a method of forming a methylammonium metal halide perovskite, wherein the cation is methylammonium.

Embodiment 4 provides the method of Embodiment 3, wherein the salt comprising the methylammonium cation is methylammonium iodide, methylammonium chloride, methylammonium bromide, or a combination thereof.

Embodiment 5 provides the method of any one of Embodiments 3-4, wherein the methylammonium metal halide is a methylammonium lead halide, a methylammonium tin halide, a methylammonium tin-lead halide, or a combination thereof.

Embodiment 6 provides the method of any one of Embodiments 3-5, wherein the methylammonium metal halide is a methylammonium lead iodide (CH₃—NH₃)PbI₃, methylammonium tin iodide (CH₃—NH₃)SnI₃, or a methylammonium tin-lead iodide (CH₃—NH₃)(Sn₁₋ₓPbₓ)I₃.

Embodiment 7 provides the method of any one of Embodiments 1-6, wherein the method is a method of forming an organic or inorganic metal halide perovskite film, wherein the hydrocarbylammonium metal halide that is cation exchanged is a hydrocarbylammonium metal halide film.

Embodiment 8 provides the method of Embodiment 7, wherein the hydrocarbylammonium metal halide film is a melt-processed hydrocarbylammonium metal halide film.

Embodiment 9 provides the method of any one of Embodiments 7-8, wherein the organic or inorganic metal halide film has an average thickness of 1 nm to 10 mm.

Embodiment 10 provides the method of any one of Embodiments 7-9, wherein the organic or inorganic metal halide film has an average thickness of about 350 nm to about 950 nm.

Embodiment 11 provides the method of any one of Embodiments 8-10, wherein the method comprises melt-processing hydrocarbylammonium metal halide at a temperature of no greater than about the melting point of the hydrocarbylammonium metal halide, to form the melt-processed hydrocarbylammonium metal halide film.

Embodiment 12 provides the method of any one of Embodiments 8-11, wherein the method comprises melt-processing hydrocarbylammonium metal halide at a temperature of no greater than 300° C., to form the melt-processed hydrocarbylammonium metal halide film.

Embodiment 13 provides the method of any one of Embodiments 8-12, wherein the method comprises melt-processing hydrocarbylammonium metal halide at a temperature of no greater than 295° C. to form the melt-processed hydrocarbylammonium metal halide film.

Embodiment 14 provides the method of any one of Embodiments 8-13, wherein the melt-processing comprises applying sufficient heat to the hydrocarbylammonium metal halide to melt the hydrocarbylammonium metal halide.

Embodiment 15 provides the method of any one of Embodiments 8-14, wherein the melt-processing comprises applying simultaneous top and bottom face heat to the hydrocarbylammonium metal halide.

Embodiment 16 provides the method of any one of Embodiments 1-15, wherein the cation exchanging comprises contacting the hydrocarbylammonium metal halide with a solution comprising the salt at sufficient temperature and for sufficient time so that a predetermined proportion of the hydrocarbylammonium cation exchanges with the organic or inorganic cation.

Embodiment 17 provides the method of Embodiment 16, wherein the solution comprises a solution of the salt in an organic solvent.

Embodiment 18 provides the method of any one of Embodiments 16-17, wherein the organic solvent is a polar organic solvent.

Embodiment 19 provides the method of Embodiment 18, wherein the organic solvent is isopropanol.

Embodiment 20 provides the method of any one of Embodiments 16-19, further comprising annealing or drying the organic or inorganic metal halide perovskite.

Embodiment 21 provides the method of any one of Embodiments 1-20, wherein the organic or inorganic metal halide is an organic or inorganic lead halide, an organic or inorganic tin halide, or a combination thereof.

Embodiment 22 provides the method of any one of Embodiments 1-21, wherein the organic or inorganic metal halide is an organic or inorganic metal iodide, an organic or inorganic metal bromide, an organic or inorganic metal chloride, an organic or inorganic metal halide comprising a mixture of two or more halides, or a combination thereof.

Embodiment 23 provides the method of any one of Embodiments 1-22, wherein the organic or inorganic metal halide is an organic or inorganic lead iodide, an organic or inorganic lead bromide, an organic or inorganic lead chloride, an organic or inorganic lead halide comprising a combination of two or more halides, an organic or inorganic tin iodide, an organic or inorganic tin bromide, an organic or inorganic tin chloride, an organic or inorganic tin halide comprising a combination of two or more halides, or a combination thereof.

Embodiment 24 provides the method of any one of Embodiments 1-23, wherein the organic or inorganic metal halide is an organic or inorganic lead iodide or an organic or inorganic tin iodide.

Embodiment 25 provides the method of any one of Embodiments 1-24, wherein the hydrocarbylammonium group is a phenyl(C₁-C₅)alkylammonium group, wherein the phenyl is unsubstituted or substituted with one or more halides.

Embodiment 26 provides the method of any one of Embodiments 1-25, wherein the hydrocarbylammonium group is an phenylethylammonium group, wherein the phenyl is unsubstituted or substituted with one or more halides.

Embodiment 27 provides the method of any one of Embodiments 1-26, wherein the hydrocarbylammonium group is phenylethylammonium (PEA), 2-fluorophenethylammonium (2-FPEA), 3-fluorophenethylammonium (3-FPEA), β-methylphenethylammonium (β-Me-PEA), or 2,3,4,5,6-pentafluorophenethylammonium (5FPEA).

Embodiment 28 provides the method of any one of Embodiments 1-27, wherein the hydrocarbylammonium metal halide is phenylethylammonium lead iodide ((PEA)₂PbI₄), phenylethylammonium tin iodide ((PEA)₂SnI₄), or phenylethylammonium lead iodide ((PEA)₂(Sn₁₋ₓPbₓ)I₄).

Embodiment 29 provides the method of any one of Embodiments 1-28, wherein the organic or inorganic metal halide perovskite is formed on a substrate.

Embodiment 30 provides the method of Embodiment 29, wherein the substrate comprises a glass substrate.

Embodiment 31 provides the method of any one of Embodiments 29-30, wherein the substrate comprises a glass substrate coated with TiO₂.

Embodiment 32 provides the method of any one of Embodiments 1-31, wherein the method is a method of forming a photovoltaic device comprising the organic or inorganic metal halide perovskite.

Embodiment 33 provides the method of Embodiment 32, wherein the photovoltaic device is a solar cell.

Embodiment 34 provides an organic or inorganic metal halide perovskite formed using the method of any one of Embodiments 1-33.

Embodiment 35 provides a photovoltaic device comprising organic or inorganic metal halide perovskite formed using the method of any one of Embodiments 1-33.

Embodiment 36 provides a method of forming a methylammonium lead iodide perovskite film, the method comprising:

melt-processing phenylethylammonium lead iodide at a temperature of no greater than 295° C., to form a melt-processed phenylethylammonium lead iodide film; and

cation exchanging the melt-processed phenylethylammonium lead iodide film with a solution of methylammonium iodide that exchanges with the phenylethylammonium cation of the phenylethylammonium lead iodide film, to form the methylammonium lead iodide perovskite film.

What is claimed is:

1. A method of forming an organic or inorganic metal halide perovskite, the method comprising:
cation exchanging a hydrocarbylammonium metal halide with a salt comprising an organic or inorganic cation that exchanges with the hydrocarbylammonium cation of the hydrocarbylammonium metal halide, to form the organic or inorganic metal halide perovskite, wherein the hydrocarbyl group is substituted or unsubstituted.

2. The method of claim 1, wherein the cation is cesium, rubidium, formamidinium, potassium, methylammonium, or a combination thereof.

3. The method of claim 1, wherein the method is a method of forming a methylammonium metal halide perovskite, wherein the cation is methylammonium.

4. The method of claim 3, wherein the salt comprising the methylammonium cation is methylammonium iodide, methylammonium chloride, methylammonium bromide, or a combination thereof.

5. The method of claim 3, wherein the methylammonium metal halide is a methylammonium lead iodide (CH₃—NH₃)PbI₃, methylammonium tin iodide (CH₃—NH₃)SnI₃, or a methylammonium tin-lead iodide (CH₃—NH₃)(Sn₁₋ₓPbₓ)I₃.

6. The method of claim 1, wherein the method is a method of forming an organic or inorganic metal halide perovskite film, wherein the hydrocarbylammonium metal halide that is cation exchanged is a hydrocarbylammonium metal halide film.

7. The method of claim 6, wherein the hydrocarbylammonium metal halide film is a melt-processed hydrocarbylammonium metal halide film.

8. The method of claim 6, wherein the organic or inorganic metal halide film has an average thickness of 1 nm to 10 mm.

9. The method of claim 7, wherein the method comprises melt-processing hydrocarbylammonium metal halide at a temperature of no greater than about the melting point of the hydrocarbylammonium metal halide, to form the melt-processed hydrocarbylammonium metal halide film.

10. The method of claim 1, wherein the cation exchanging comprises contacting the hydrocarbylammonium metal halide with a solution comprising the salt at sufficient temperature and for sufficient time so that a predetermined proportion of the hydrocarbylammonium cation exchanges with the organic or inorganic cation.

11. The method of claim 10, wherein the solution comprises a solution of the salt in an organic solvent.

12. The method of claim 1, wherein the organic or inorganic metal halide is an organic or inorganic lead halide, an organic or inorganic tin halide, an organic or inorganic tin-lead halide, or a combination thereof.

13. The method of claim 1, wherein the organic or inorganic metal halide is an organic or inorganic metal iodide, an organic or inorganic metal bromide, an organic or inorganic metal chloride, an organic or inorganic metal halide comprising a mixture of two or more halides, or a combination thereof.

14. The method of claim 1, wherein the hydrocarbylammonium group is a phenyl(C₁-C₅)alkylammonium group, wherein the phenyl is unsubstituted or substituted with one or more halides.

15. The method of claim 1, wherein the hydrocarbylammonium group is phenylethylammonium (PEA), 2-fluorophenethylammonium (2-FPEA), 3-fluorophenethylammonium (3-FPEA), β-methylphenethylammonium (β-Me-PEA), or 2,3,4,5,6-pentafluorophenethylammonium (5FPEA).

16. The method of claim 1, wherein the hydrocarbylammonium metal halide is phenylethylammonium lead iodide ((PEA)₂PbI₄), phenylethylammonium tin iodide ((PEA)₂SnI₄), or phenylethylammonium tin-lead iodide ((PEA)₂(Sn₁₋ₓPbₓ)I₄).

17. The method of claim 1, wherein the organic or inorganic metal halide perovskite is formed on a substrate comprising a glass substrate coated with TiO₂.

18. The method of claim 1, wherein the method is a method of forming a photovoltaic device comprising the organic or inorganic metal halide perovskite.

19. The method of claim 18, wherein the photovoltaic device is a solar cell.

20. A method of forming a methylammonium lead iodide perovskite film, the method comprising:
melt-processing phenylethylammonium lead iodide at a temperature of no greater than 295° C., to form a melt-processed phenylethylammonium lead iodide film; and
cation exchanging the melt-processed phenylethylammonium lead iodide film with a solution of methylammonium iodide that exchanges with the phenylethylammonium cation of the phenylethylammonium lead iodide film, to form the methylammonium lead iodide perovskite film.
